(19) United States
(12) Patent Application Publication
(10) Pub. No.: US 2010/0191205 A1
(43) Pub. Date: Jul. 29, 2010

(54) OPENING SYSTEM FOR ABSORBENT ARTICLES

(75) Inventors: Raquel Carbonari, Philadelphia, PA (US); Joshua Carney, Philadelphia, PA (US); Bernice Cator, Willingboro, NJ (US)

Correspondence Address: DRINKER BIDDLE & REATH (DC), 1500 K STREET, N.W., SUITE 1100, WASHINGTON, DC, 20005-1209, US

(73) Assignee: SCA HYGIENE PRODUCTS AB, Goteborg (SE)

(21) Appl. No.: 12/361,972

(22) Filed: Jan. 29, 2009

(51) Int. Cl. A61F 13/51

(52) U.S. Cl. 604/359

(57) ABSTRACT

An absorbent article includes a topsheet, a backsheet, and an absorbent core disposed between the topsheet and backsheet. A first barrier layer and a second barrier encapsulate an additive therebetween. The second barrier layer is disposed between the topsheet and the absorbent core and the first barrier layer is disposed on a topsheet. An opening system is provided that allows the first barrier layer to be secured to a release paper, outer packaging or article itself, so that it is removed with the release paper, outer packaging or unfolding of the product. As such, the first barrier layer need not be separately removed, and is hidden from view, thereby eliminating the step of separately removing the barrier layer.

### FIELD OF THE INVENTION

The present invention relates to an opening system for absorbent articles. More particularly, the present invention relates to an opening system for an absorbent article having a moisture-sensitive additive.

### BACKGROUND OF THE INVENTION

There are many types of absorbent articles, such as feminine hygiene products, incontinence guards, infant diapers, and training pants. Absorbent articles typically include a topsheet facing the body of the user, a backsheet facing the garment of a user, and an absorbent layer sandwiched between the inner, topsheet and outer, backsheet.

Various additives may be included in absorbent products for different reasons. For example, odor-inhibiting additives or deodorants may be added, such as zeolites and silica. Another type of additive is a softener or lotion, which is transferred from the product to the wearer's skin. Softeners may be used in adult incontinence guards, diapers, training pants, feminine hygiene products, and the like.

In addition, probiotic and prebiotic bacteria may be added, such as lactic acid producing bacteria belonging to the genus Lactobacillus. Lactobacilli has an antagonistic effect against pathogenic microbial species. Use of this additive has shown to treat and effectively prevent microbial infections. The use of lactobacilli as an additive is described, for example, in WO 2007/073263 and WO 2008/054268.

The additives described above lose some of their effectiveness when exposed to varying degrees of moisture and light. Therefore, effort has been made to effectively enclose the additives to prevent their deterioration. For example, WO 2008/054268 describes a delivery system where the additive is enclosed between two impervious layers. In one embodiment, the layers are made from a metal foil. As described therein, one of the barrier layers is placed above the topsheet and the other layer is placed below the topsheet with the additive sealed therebetween. The barrier layer placed above the topsheet is exposed to the user. To activate the additive, the top barrier layer must be removed by the user. However, a drawback is that an additional step of removing the barrier layer becomes necessary. In addition, the additive may unnecessarily contact the hand of the user.

### SUMMARY

According to a first aspect of the present invention, an absorbent article includes a topsheet, backsheet, an absorbent core disposed between the topsheet and backsheet. A first barrier layer and a second barrier encapsulate an additive therebetween. The second barrier layer is disposed between the topsheet and the absorbent core and the first barrier layer is disposed between the topsheet and a release paper. The first barrier layer is attached to the release paper so that the first barrier layer is removed upon removal of the release paper.

According to a second aspect of the present invention, an absorbent article includes a a topsheet, a backsheet, and an absorbent core disposed between the topsheet and backsheet. A first barrier layer and a second barrier encapsulate an additive therebetween. The second barrier layer is disposed between the topsheet and the absorbent core and the first barrier layer is disposed between a topsheet and an outer packaging. The first barrier layer is attached to the outer packaging so that it is released from the topsheet of the article upon unfolding of the article.

According to a third aspect of the present invention, an absorbent article includes a topsheet, a backsheet, and an absorbent core disposed between the topsheet and backsheet. A securing mechanism for securing the article around the waist of the user is provided. A first barrier layer and a second barrier encapsulate an additive therebetween. The second barrier layer is disposed between the topsheet and the absorbent core and the first barrier layer is disposed between a topsheet and the securing mechanism. The first barrier layer is attached to the securing mechanism so that the first barrier layer is released from the topsheet of the article upon unfolding of the article

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements.

FIG. 1 is a plan view of an absorbent article of the prior art with a separately removable protective barrier sheet.

FIG. 2 is a perspective view of a first embodiment of the present invention with the absorbent article in a wrapped position.

FIG. 3 is a perspective view of the first embodiment of the present invention with the release paper and a foil being removed in one action.

FIG. 4 is a perspective view of an absorbent article with portions removed including an additive in accordance with the features of the present invention.

FIG. 5 is a cross sectional view of the first embodiment of the present invention where the absorbent article is in a wrapped position.

FIG. 6 is an exploded view of an absorbent article showing various layers and including an additive and accompanying barrier layers.

FIG. 7 is a perspective view of a second embodiment of the present invention, wherein a first end section the absorbent article is unfolded.

FIG. 8 is a perspective view of the second embodiment of FIG. 7 of the present invention, wherein the second end section of the absorbent article is unfolded thereby releasing the barrier layer.

FIG. 9 is a cross sectional view of the second embodiment of the present invention shown in FIG. 7 as taken along line 9-9.

FIG. 10 is a cross sectional view of the second embodiment of the present invention shown in FIG. 7 as taken along line 10-10.

FIG. 11 is a perspective view of the third embodiment of the present invention similar to the second embodiment, except with no wings.

FIG. 12 is a perspective view of a fourth embodiment of the present invention, wherein first and second end sections of the absorbent article are unfolded.

FIG. 13 is a perspective view of the fourth embodiment of the present invention as shown in FIG. 12 herein a first side section is unfolded.

FIG. 14 is a perspective view of the fourth embodiment of the present invention as shown in FIG. 12 wherein a second side section is unfolded.

FIG. 15 is a cross sectional view of the fourth embodiment of the present invention as shown in FIG. 12 and taken along line 15-15.

FIG. 16 is a perspective view of the fifth embodiment of the present invention similar to the fourth embodiment, except with no wings.

FIG. 17 is a perspective view of a sixth embodiment of the present invention wherein the absorbent article is in a folded position.

FIG. 18 is a perspective view of the sixth embodiment of the present invention wherein a first end section of the absorbent article is unfolded.

FIG. 19 is a perspective view of the sixth embodiment of the present invention wherein a second end section of the absorbent article is unfolded from a first middle section of the absorbent article.

FIG. 20 is a perspective view of the sixth embodiment of the present invention wherein the second end section of the absorbent article is unfolded from the second middle section of the absorbent article.

FIG. 21 is a perspective view of the garment facing side of the absorbent article of the sixth embodiment of the present invention in an unfolded position.

FIG. 22 is a cross sectional view of the sixth embodiment of the present invention as shown in FIG. 17 and taken along line 22-22.

FIG. 23 is a perspective view of a seventh embodiment of the present invention in a folded position.

FIG. 24 is a perspective view of the seventh embodiment of the present invention wherein a first end section of the absorbent article is unfolded.

FIG. 25 is perspective view of the seventh embodiment of the present invention wherein a second end section of the absorbent article is unfolded.

FIG. 26 is a perspective view of the seventh embodiment of the present invention wherein first and second side sections are unfolded.

FIG. 27 is a cross sectional view of the seventh embodiment of the present invention as shown in FIG. 23 and taken along line 27-27.

### DETAILED DESCRIPTION OF THE PREFERRED EMBODIMENTS

The present invention relates to an opening system for absorbent articles including delivery systems for the delivery of various additives, such as skin caring agents, odor inhibiting substances and probiotic bacteria. An absorbent article or garment is defined as an article or garment used for the absorption of body fluids, including but not limited to, infant diapers and training pants, adult incontinence products, feminine hygiene products, gender specific absorbent products, and pet training absorbent articles. However, an absorbent article may also include, but is not limited to, other types of products, such as wet wipes, dry wipes, or the like.

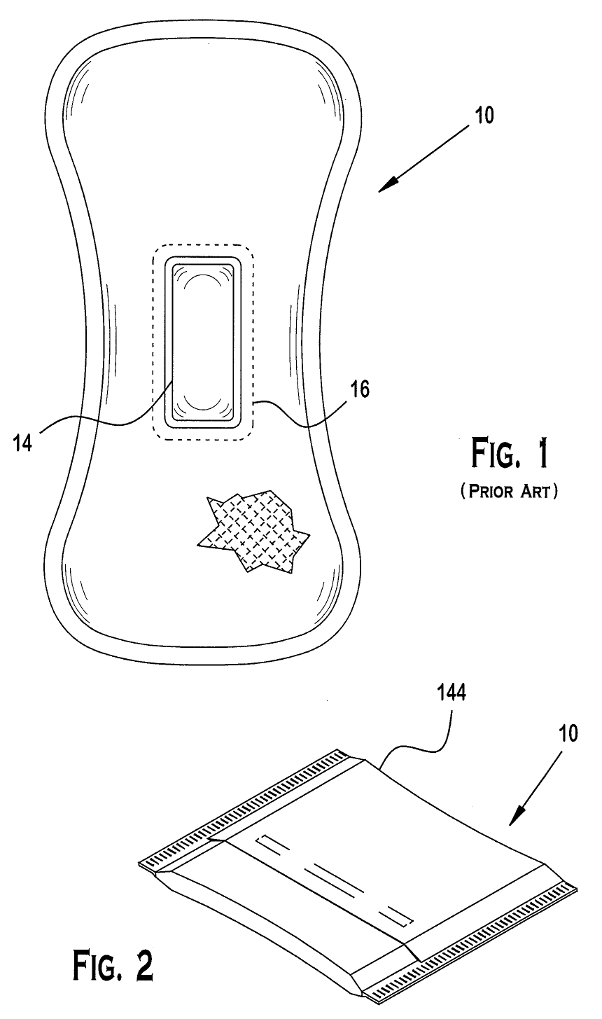

As described above, many of the additives require varying degrees of moisture to maintain their viability. As such, prior art systems have been devised that seal the additive in a relatively moisture free environment. For example, with reference to FIG. 1, an absorbent article 10 may include an additive (not shown) to be sealed between a first barrier layer 14 and a second barrier layer 16. However, one having ordinary skill in the art would understand that there are alternative ways of placing and sealing an additive on an absorbent article, depending on application and design preference.

Preferably, the first barrier layer 14 is placed on a user facing side of the absorbent article 10. The first barrier layer 14 and second barrier layer 16 are sealed together along their edges. Suitable sealing methods include, but are not limited to, ultrasonic welding, heat sealing, heating sealing at low temperatures, and cold sealing. The first barrier layer 14 and the second barrier layer 16 may also be glued together. The first barrier layer 14 and second barrier layer 16 may have any suitable shape, such as circular, rectangular, square, oval, or irregular. The first barrier layer 14 and second barrier layer 16 may be made of the same or different material, depending on application and design preference.

For example, the first barrier layer 14 and second barrier layer 16 may be water vapor impermeable. The first barrier layers 14 and 16 may be made, for example, from a metal foil, polymeric film, a laminate of a metal foil, polymeric film and/or a wax. In addition, a metal oxide layer and a silicon oxide layer may be used. Examples of the materials that can be used as the barrier layers are found in WO 2008/054268, which is incorporated by reference herein.

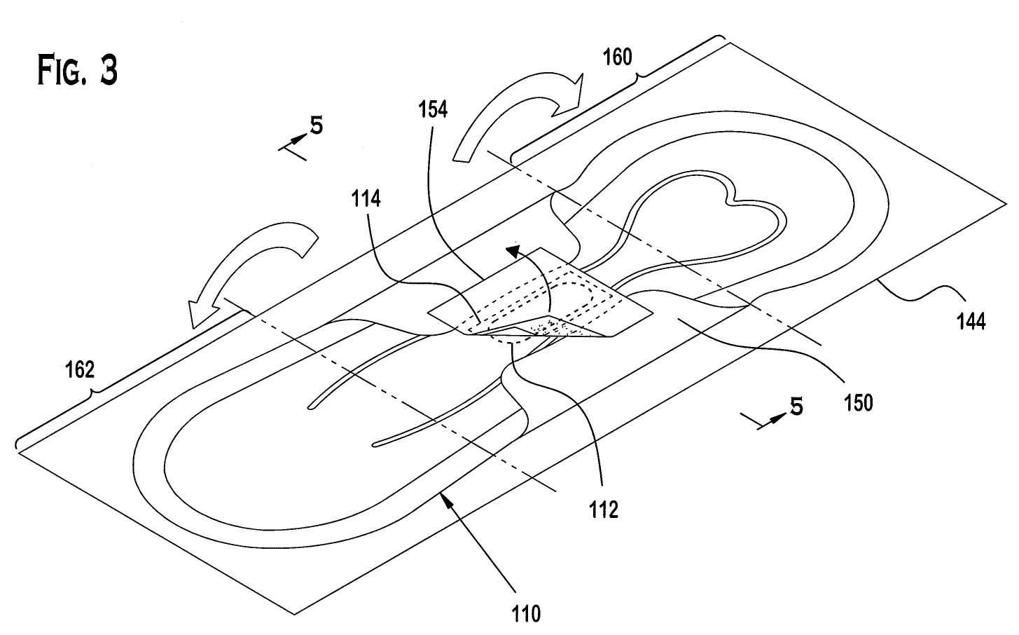

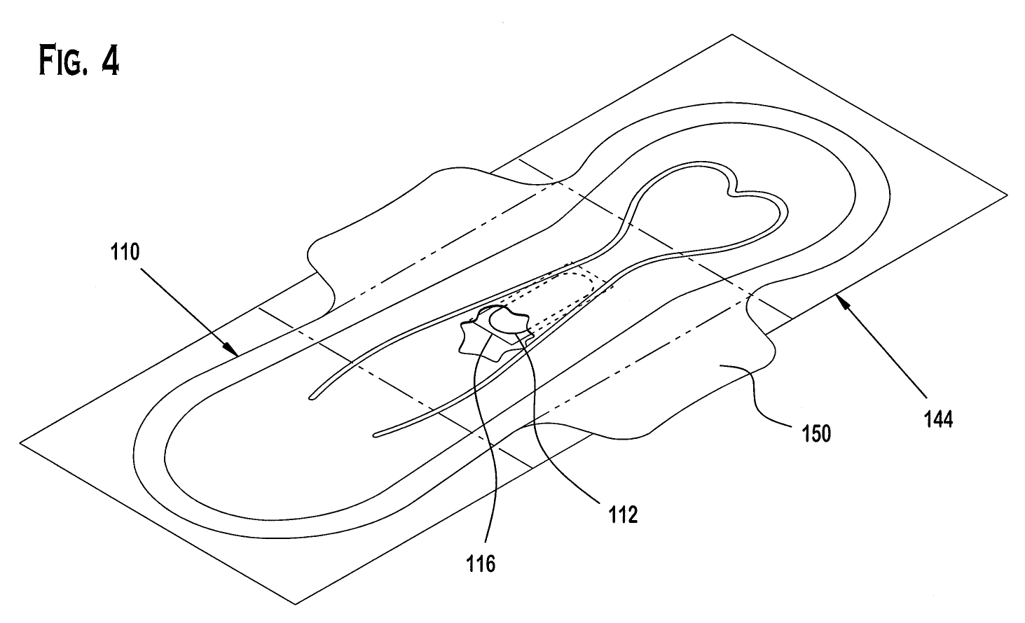

With reference to FIGS. 2-6, an absorbent article 110 according to a first embodiment of the present invention will be described in more detail. The absorbent article 110 includes an additive 112 that is positioned between a first barrier layer 114 and a second barrier layer 116, as shown, for example, in FIGS. 3-6. The additive 112 may include any substance for improved health and hygiene for the user of the article, such as odor-inhibiting additives or deodorants, softeners, lotions, and oils, as well as probiotic and prebiotic bacteria. Preferably, the probiotic bacteria are lactic acid producing bacteria belonging to the genus Lactobacillus. Lactobacilli has an antagonistic effect against pathogenic microbial species.

Other examples of additives include but are not limited to pH regulation substances, such as buffered citric acid; humidity stabilization substances, such as Carbamide; botanical substances, such as Aloe Vera; antioxidants, such as ascorbic acid; vitamins, skin soothing substances, and hydrophobic substances, such as olive oil. Antibacterial substances, such as ZnO, and antifungal substances, may also be used. Other additives for comfort and pleasure may include cooling substances, anti itching substances, aromatic substances, and chemicals providing sensory stimuli, such as making the absorbent article feel wet. Odor inhibiting substances may include zeolites and silica.

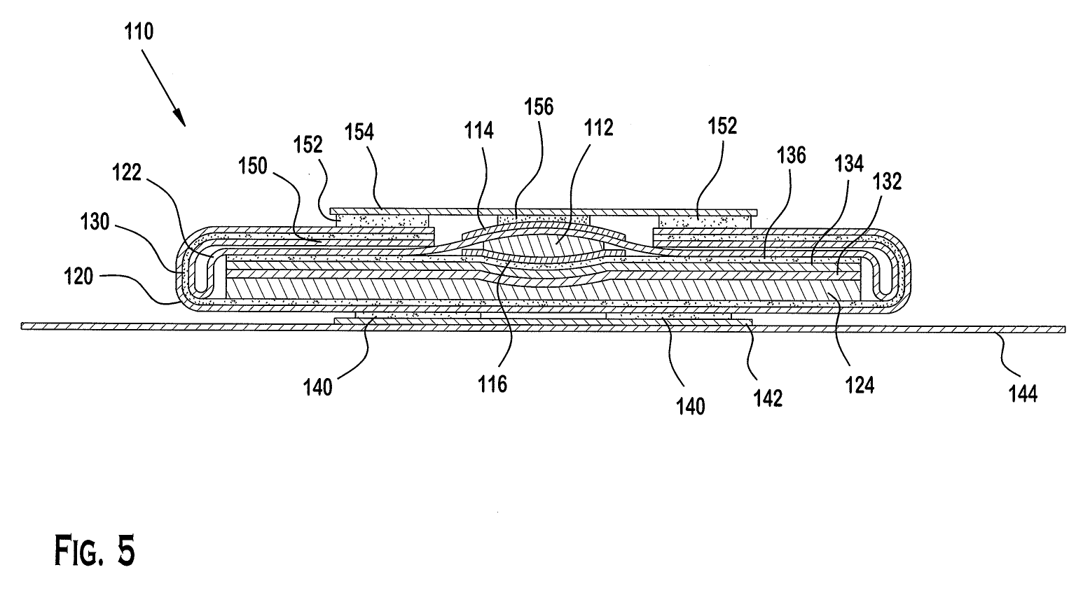

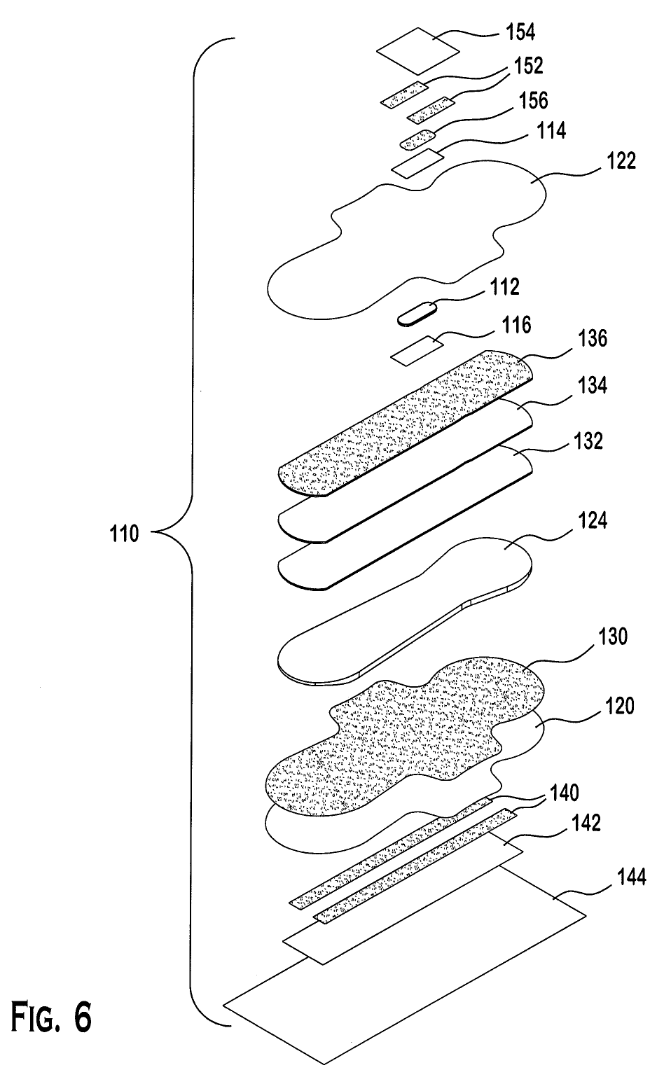

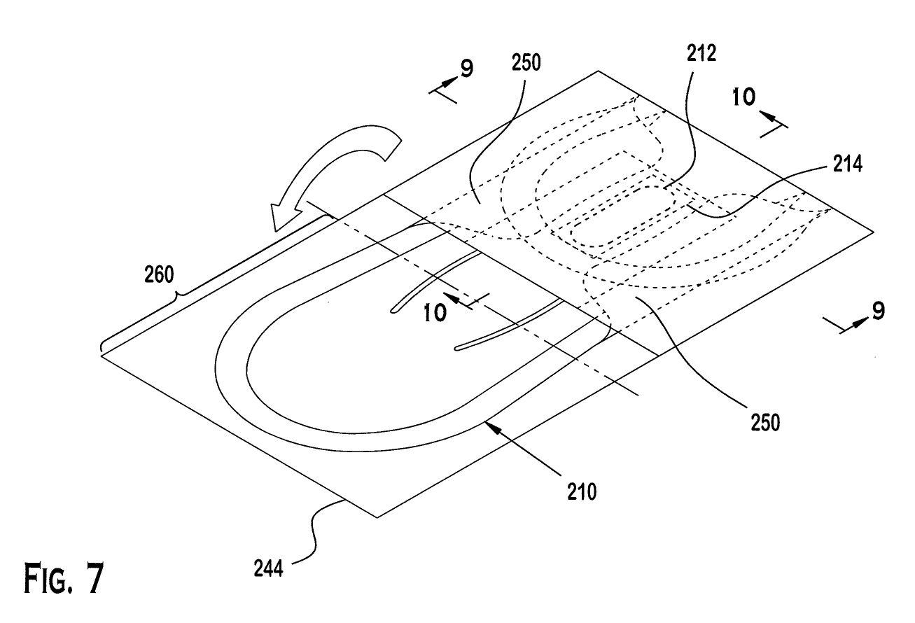

As shown in FIGS. 5-6, the absorbent article 110 may be made from various layers of material. For example, the absorbent article 110 shown therein includes a back sheet 120 and a top sheet 122 which sandwich an absorbent core 124 therebetween. Preferably, the backsheet 120 and top sheet 122 are connected via a layer of glue 130. However, it should be understood that the backsheet 120 and topsheet 122 may be connected in other ways known in the art. Numerous other layers may also be included, but are not necessary, such as an additional absorbent core 132, a transfer layer 134, and a layer of glue 136 to connect the transfer layer 134 to the top sheet 122. However, the specific layers used may vary, depending on design preference and application. For example, other layers not shown may be used, such as an additional back sheet, or any other intervening layer, such as SMS (Spunbond MeltBlown Spunbond) SSS (there layers of Spunbond), Carded NW, Hydroentangled, and the like. Likewise, the particular number and order of layers is optional, depending on application and design preference. In addition, certain layers shown in the drawings and described in the specification may be eliminated.

With further reference to FIGS. 5 and 6, the absorbent article 110 also preferably includes an adhesive 140 disposed on the garment facing side of the back sheet 120 of the absorbent article 110, to adhere the absorbent article 110 to the garment of the user. Preferably, the adhesive 140 is a pressure sensitive hot melt. However, it should be understood that the adhesive may be any other securing mechanism, as is known in the art. In addition, the adhesive 140 is optional, particularly with regard to larger adult incontinence guards. If an adhesive 140 is included, a release paper 142 may be provided so that the absorbent article 110 does not stick to an outer wrapping or packaging 144, and may be freely removable therefrom. Alternatively, the adhesive 140 may be directly connected to the outer wrapping 144. In this case, the outer packaging acts as a release paper for the garment facing side of the absorbent article 110, and must be peeled away prior to use.

With reference to FIGS. 3, 5 and 6, the back sheet 120 of the absorbent article 110 may include a pair of wings 150 to be connected on the side edges of the garment of the user to thereby protect the garment from leaks, as is known in the art. With reference to FIG. 5, the wings 150 include an adhesive 152 applied thereon so that the wings 150 adhere to the outer side of the garment of the user, thereby protecting the side edges of the garment of the user. Preferably, the adhesive 152 is a pressure sensitive adhesive, but may include other types of adhesive or securing mechanisms.

As best shown in FIG. 3, the wings 150 are preferably folded onto the top side of the absorbent article 110, and include a release paper 154 placed thereon. In the prior art, the release paper had to be removed first, and then the first barrier layer became exposed for removal separate from the release paper. However, in accordance with the features of the present invention, the release paper 154 is directly attached to the first barrier layer 114 so that the first barrier layer 114 is removed upon removal of the release paper 154.

As shown in FIGS. 5 and 6, the first barrier layer 114 is preferably attached to the release paper 154 by a bead of glue 156. Thereafter, the first barrier layer 114 and second barrier layer 116 may be sealed together, as described above. However, it should be understood that the first barrier layer 114 may be attached to the release paper 154 by other means, such as by an adhesive covering the entire surface of the first barrier layer 114 facing the release paper 154, by use of a pressure sensitive adhesive, ultrasonic bonding, or by any other securing mechanism known in the art, depending on application and design preference.

With reference to FIGS. 2-4, operation of the opening system of the present invention will be described in more detail. As described above, the absorbent article 110 may include an outer packaging 144, as shown in FIG. 2. As shown in FIG. 3, a first end section 160 is unfolded in the direction of the indicated arrow, while a second end section 162 is unfolded in the direction of the indicated arrow. The release paper 154 may then be removed from the wings 150. Upon removal of the release paper 154, the first barrier layer 114 is also removed. According to features of the present invention, the first barrier layer 114 need not be separately removed, and is hidden from view, thereby eliminating the step of separately removing the barrier layer.

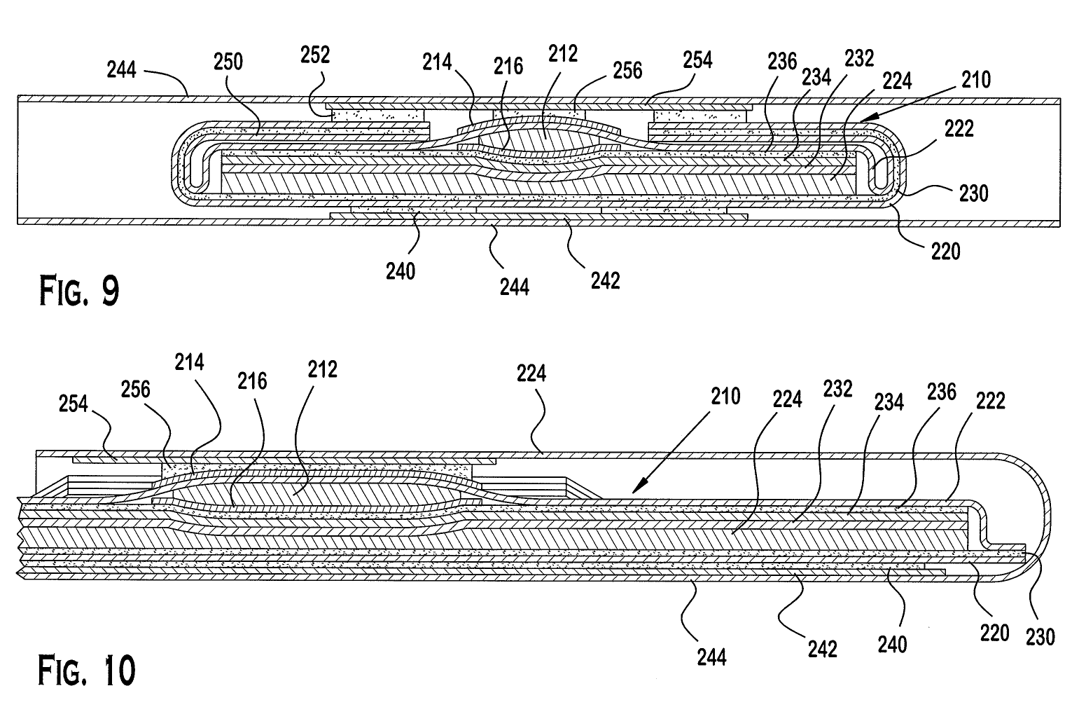

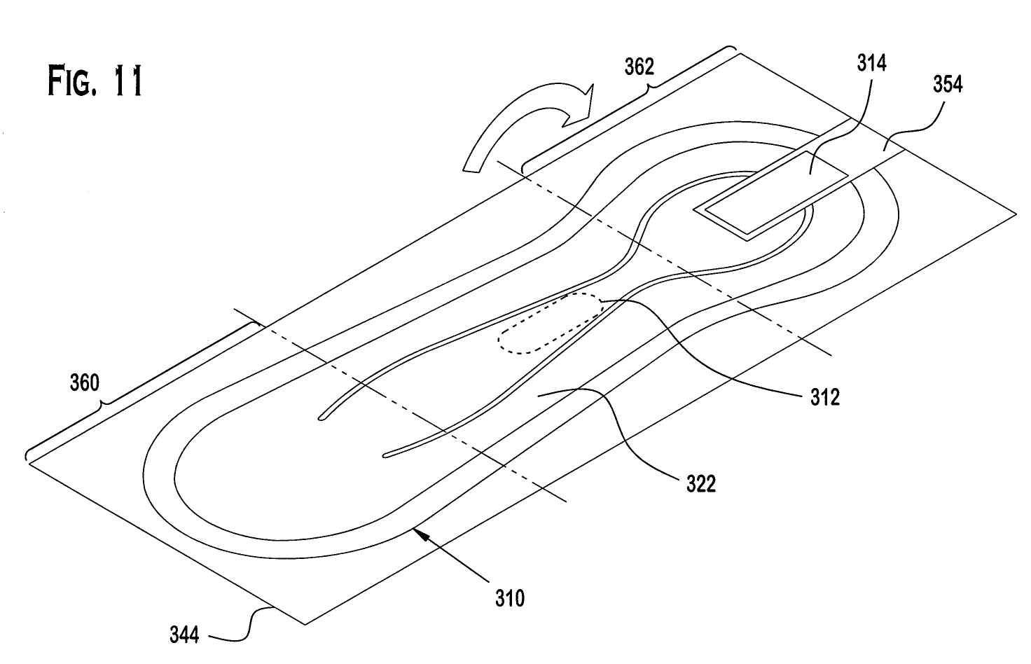

With reference to FIGS. 7-10, an absorbent article 210 according to a second embodiment of the present invention will be described in more detail. With reference to FIGS. 9 and 10, the absorbent article 210 includes an additive 212 that is positioned between a first barrier layer 214 and a second barrier layer 216. The additive 212 corresponds to the same type of additive described above with respect to additive 112 of the first embodiment.

Like the first embodiment, the absorbent article 210 may be made from various layers of material. For example, the absorbent article 210 shown therein includes a back sheet 220 and a top sheet 222 which sandwich an absorbent core 224 therebetween. Numerous other layers may also be included, such as a glue layer 230, an additional absorbent core 232, a transfer layer 234, and a glue layer 236 for adhering the transfer layer 234 to the topsheet 222. However, the specific layers used may vary, depending on design preference and application. For example, additional layers not shown the drawings may be used, while certain layers shown in the drawings and described in the specification may be eliminated.

With further reference to FIGS. 9 and 10, the absorbent article 210 also preferably includes an adhesive 240 disposed on the garment facing side of the back sheet 220 of the absorbent article 210, to adhere the absorbent article 210 to the garment of the user. Preferably, the adhesive 240 is a pressure sensitive hot melt. However, it should be understood that the adhesive may be any other securing mechanism, as is known in the art. In addition, the adhesive 240 is optional, particularly with regard to larger adult incontinence guards. If an adhesive 240 is included, a release paper 242 may be provided so that the absorbent article 210 does not stick to an outer wrapping or packaging 244, and may be freely removable therefrom. Alternatively, release paper 242 may also be connected to the outer packaging 244. In addition, the adhesive 240 may be directly connected to the outer wrapping 244. In this case, the outer packaging acts as a release paper for the garment facing side of the absorbent article 210, and must be peeled away prior to use.

With reference to FIGS. 7-10, the absorbent article 210 may include a pair of wings 250 to be connected on the outer side of the garment of the user, as is known in the art. As shown in FIG. 9, the wings 250 include an adhesive 252 applied thereon so that the wings 250 adhere to the outer side of the garment of the user, thereby protecting the side edges of the garment of the user. Preferably, the adhesive 252 is a pressure sensitive adhesive, but may include other types of adhesive or securing mechanisms.

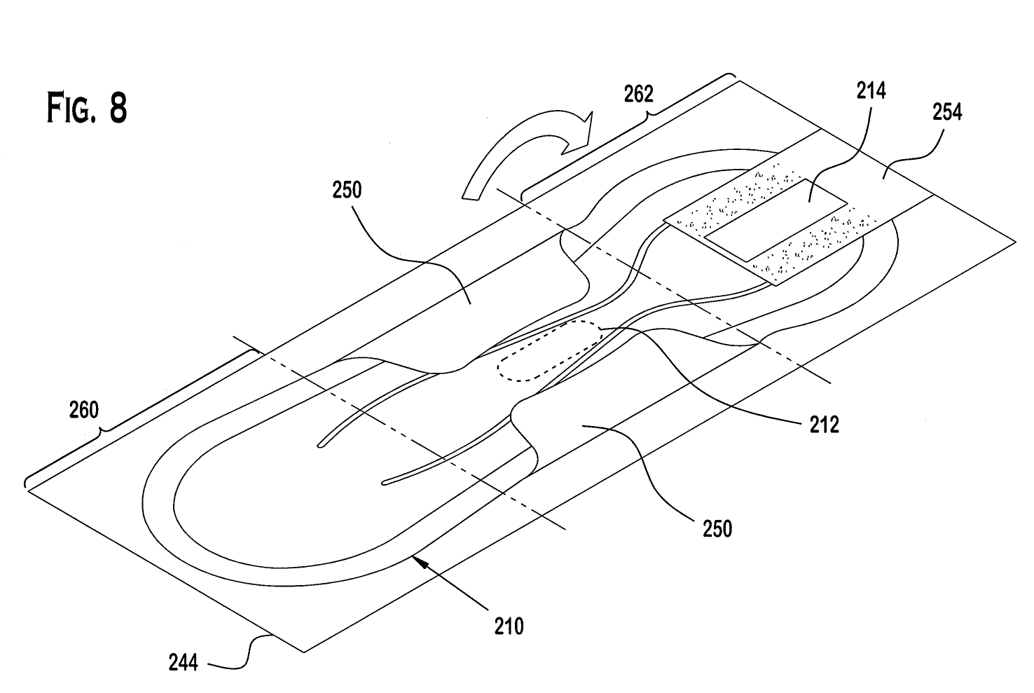

As best shown in FIG. 8, the wings 250 are preferably folded onto the top side of the absorbent article 210. However, in accordance with the features of the second embodiment of the present invention, the release paper 254 is directly connected to the outer packaging 244, by way of a glue, adhesive or any other securing mechanism. The first barrier layer 214 is then attached to the release paper 254, by way of a bead of glue 256 as shown, for example in FIGS. 9 and 10. The first barrier layer 214 and second barrier layer 216 may then be sealed together, as shown in FIGS. 9 and 10. However, it should be understood that the first barrier layer 214 may be attached to the release paper 254 by other means, such as by an adhesive covering the entire surface of the first barrier layer 214 facing the release paper 254, by use of a pressure sensitive adhesive, by ultrasonic bonding, or any other securing mechanism, depending on application and design preference.

With reference to FIGS. 7-8, operation of the opening system according to the second embodiment of the present invention will be described in more detail. As described above, the absorbent article 210 includes an outer packaging 244. As shown in FIG. 7, a first end section 260 is unfolded in the direction of the indicated arrow. At this position, the release paper 254 and first barrier layer 214 are still adhered to the absorbent article 210. As shown in FIG. 8, a second end section 262 is opened in the direction of the indicated arrow, whereupon the release paper 254 and first barrier layer 214 are removed from the wings 250. According to this embodiment, the release paper 254 and first barrier layer 214 are automatically opened upon opening of the second section 262 of the absorbent article 210. The outer packaging 244 may then be released to thereby apply the absorbent article 210 to the garment of a user.

With reference to FIG. 11, the absorbent article 310 in accordance with a third embodiment of the present invention is similar to the second embodiment, except that wings are not provided. In accordance with this embodiment, a release paper 354 is attached to the outer edge of the outer packaging 344. The first barrier layer 314 is then attached to the release paper 354. Like the second embodiment, the specific layers used may vary, depending on application and design preference. For example, additional layers not shown the drawings may be used, while certain layers shown in the drawings and described in the specification may be eliminated.

Opening of the absorbent article 310 is similar to the second embodiment. In particular, a first section 360 is unfolded, followed by the unfolding of the second section 362, which thereby releases the first barrier layer 314 from the top sheet 322 of the absorbent article 310 exposing the additive 312. The outer packaging 344 may then be released to thereby apply the absorbent article 310 to the garment of a user.

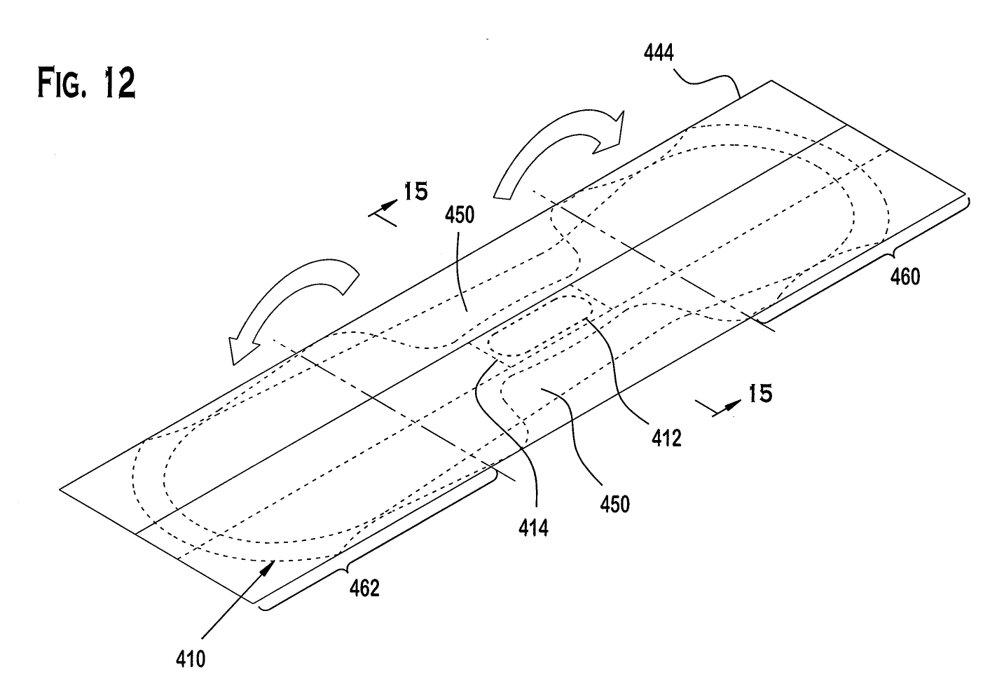

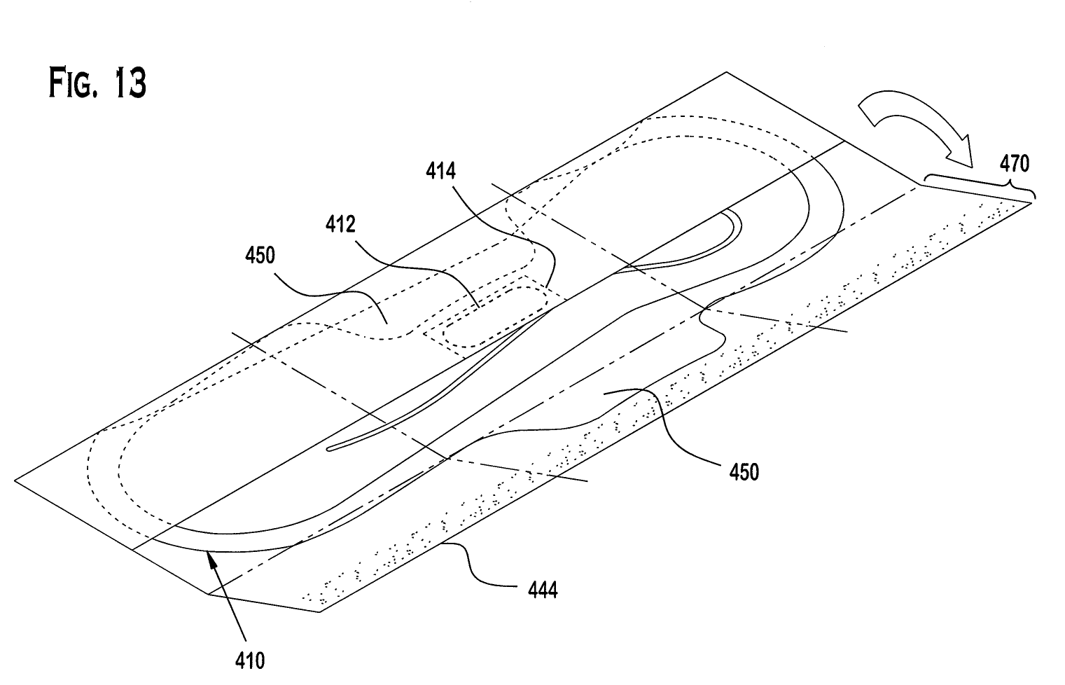

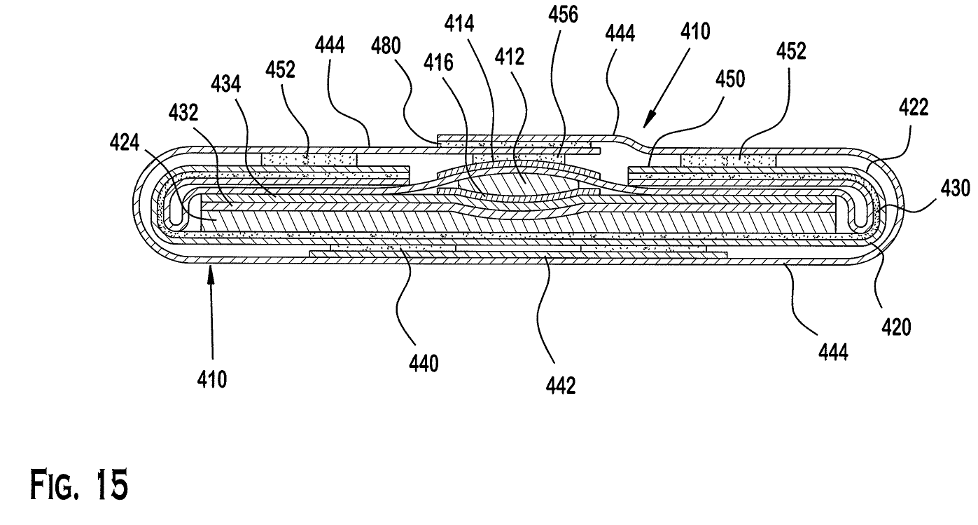

With reference to FIGS. 12-15, an absorbent article 410 according to a fourth embodiment of the present invention will be described in more detail. With reference to FIG. 15, the absorbent article 410 includes an additive 412 that is positioned between a first barrier layer 414 and a second barrier layer 416. The additive 412 corresponds to the same type of additive described above with respect to additive 112 of the first embodiment.

Like the first embodiment, the absorbent article 410 may be made from various layers of material. For example, the absorbent article 410 shown therein includes a back sheet 420 and a top sheet 422 which sandwich an absorbent core 424 therebetween. Numerous other layers may also be included, such as a glue layer 430, an additional absorbent core 432, and a transfer layer 434. However, the specific layers used may vary, depending on application and design preference. For example, additional layers not shown the drawings may be used, while certain layers shown in the drawings and described in the specification may be eliminated.

With reference to FIG. 15, the absorbent article 410 also preferably includes an adhesive 440 disposed on the garment facing side of the back sheet 420 of the absorbent article 410, to adhere the absorbent article 410 to the garment of the user. Preferably, the adhesive 440 is a pressure sensitive hot melt. However, it should be understood that the adhesive may be any other securing mechanism, as is known in the art. In addition, the adhesive 440 is optional, particularly with regard to larger adult incontinence guards. If an adhesive 440 is included, a release paper 442 may be provided so that the absorbent article 410 does not stick to an outer wrapping or packaging 444, and may be freely removable therefrom. Alternatively, release paper 442 may also be connected to the outer packaging 444. In addition, the adhesive 440 may be directly connected to the outer wrapping 444. In this case, the outer packaging acts as a release paper for the garment facing side of the absorbent article 410, and must be peeled away prior to use.

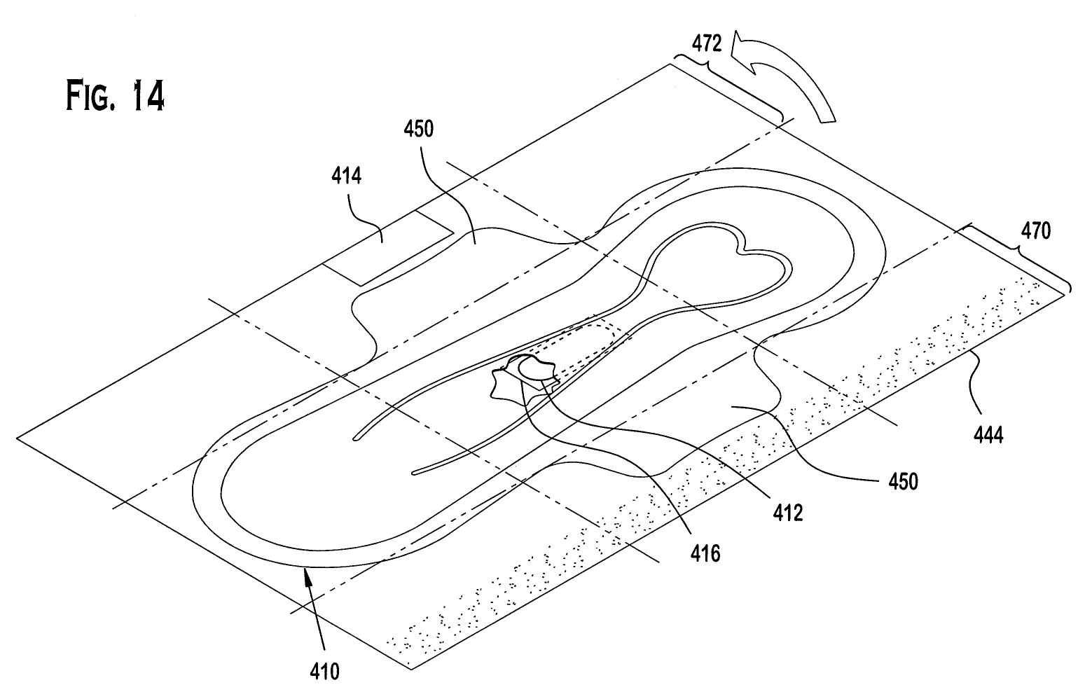

With reference to FIG. 15, the back sheet 420 of the absorbent article 410 may include a pair of wings 450 to be connected on the outer side of the garment of the user, as is known in the art. As shown in FIG. 14, the wings 450 are directly connected to the outer packaging 444 by way an adhesive 452. Preferably, the adhesive 452 is a pressure sensitive adhesive, but may include other types of adhesive.

As best shown in FIG. 12, the wings 450 are preferably folded onto the top side of the absorbent article 410. However, in accordance with the features of the fourth embodiment of the present invention, the first barrier layer 414 is directly connected to the outer packaging 444, by way of a glue, adhesive or the like, as best shown in FIG. 14.

With reference to FIGS. 12-14, operation of the opening system according to the fourth embodiment of the present invention will be described in more detail. As described above, the absorbent article 410 includes an outer packaging 444. As shown in FIG. 12, a first end section 460 and second end section 462 are opened as indicated by the respective arrows. At this position, the first barrier layer 414 is still adhered to the absorbent article 410. As shown in FIG. 13, a first side section 470 is opened as indicated by the respective arrow. Again, in this position, the first barrier layer 414 is still adhered by the absorbent article 410. With reference to FIG. 14, a second side section 472 is opened as indicated by the respective arrow, whereby the first barrier layer 414 is automatically released. The outer packaging 444 may then be released to thereby apply the absorbent article 410 to the garment of a user. As shown in FIG. 15, an overhang of the outer packaging 444 allows the outer packaging to be adhered to itself via an adhesive 480, glue or the like.

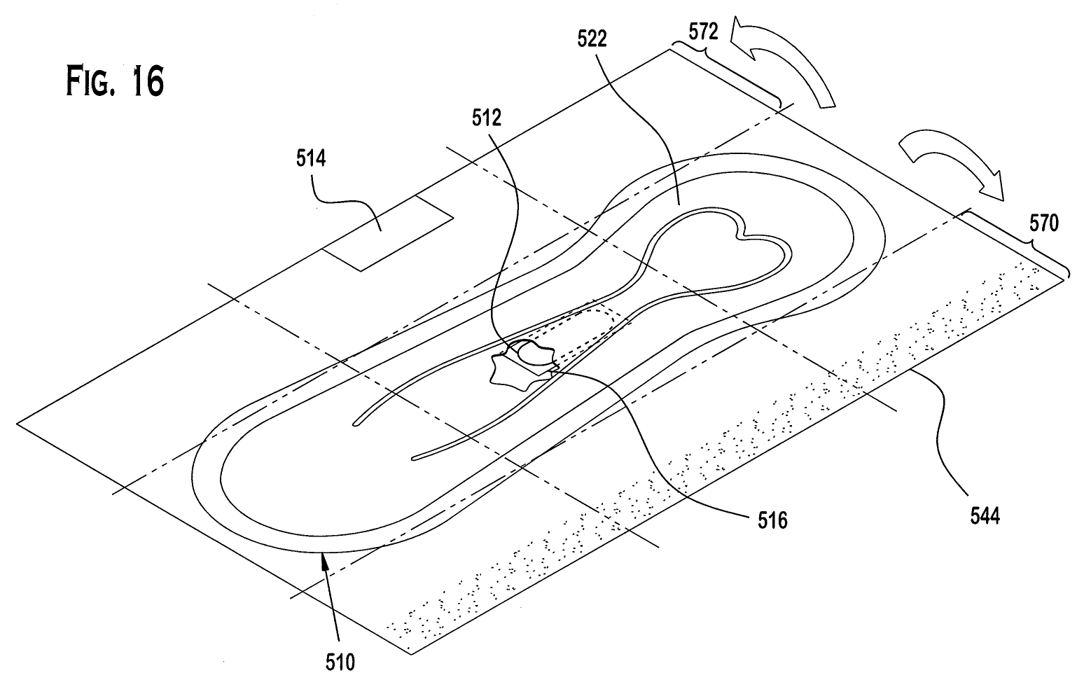

With reference to FIG. 16, an absorbent article 510 in accordance with a fifth embodiment of the present invention is similar to the fourth embodiment, except that wings are not provided. It should be understood that the specific layers used may vary, depending on application and design preference. For example, additional layers not shown the drawings may be used, while certain layers shown in the drawings and described in the specification may be eliminated. Similar to the fourth embodiment, an additive 512 is placed between a first barrier layer 514 and second barrier layer 516, and the first barrier layer 514 is directly attached to the outer packaging 544.

Opening of the absorbent article 510 is similar to the fourth embodiment. In particular, a first side section 570 and a second side section 572 are opened as indicated by the respective arrows. Upon opening of the second side section 572, the first barrier layer 514 is automatically released from the top sheet 522 of the absorbent article 510. The outer packaging 544 may then be released to thereby apply the absorbent article 510 to the garment of a user.

With reference to FIGS. 17-22, an absorbent article 610 according to a sixth embodiment of the present invention will be described in more detail. The absorbent article 610 includes an additive 612 that is positioned between a first barrier layer 614 and a second barrier layer 616. The additive 612 corresponds to the same type of additive described above with respect to additive 112 of the first embodiment.

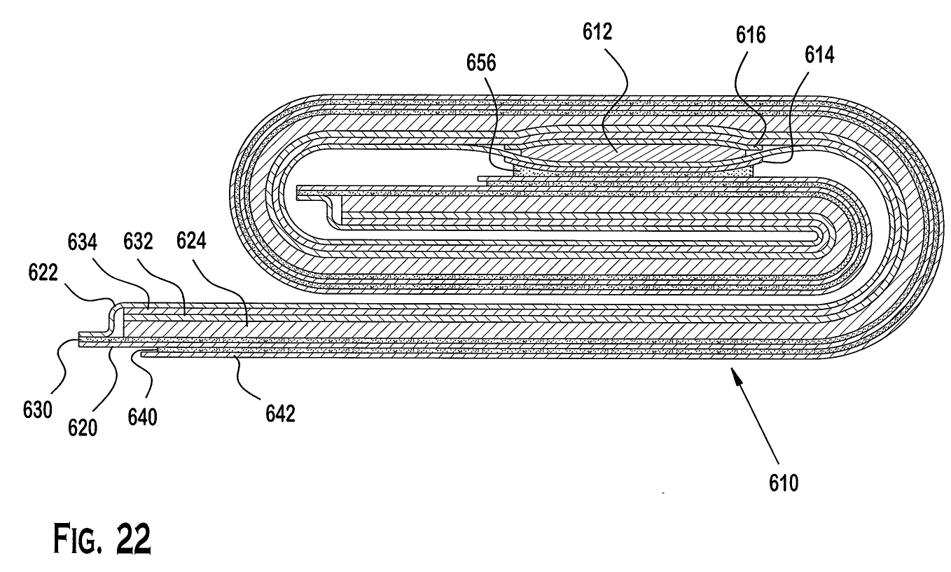

Like the first embodiment, the absorbent article 610 may be made from various layers of material. For example, the absorbent article 610 shown therein includes a back sheet 620 and a top sheet 622 which sandwich an absorbent core 624 therebetween, as shown in FIG. 22. Numerous other layers may also be included, such as a glue layer 630, an additional absorbent core 632, and a transfer layer 634. However, it should be understood that the specific layers used may vary, depending on application and design preference. For example, additional layers not shown the drawings may be used, while certain layers shown in the drawings and described in the specification may be eliminated.

The absorbent article 610 also preferably includes an adhesive 640 disposed on the garment facing side of the back sheet 620 of the absorbent article 610, to adhere the absorbent article 610 to the garment of the user. Preferably, the adhesive 640 is a pressure sensitive hot melt or any other securing mechanism. A release paper 642 is provided adjacent the adhesive 640 to prevent the adhesive from sticking prior to use.

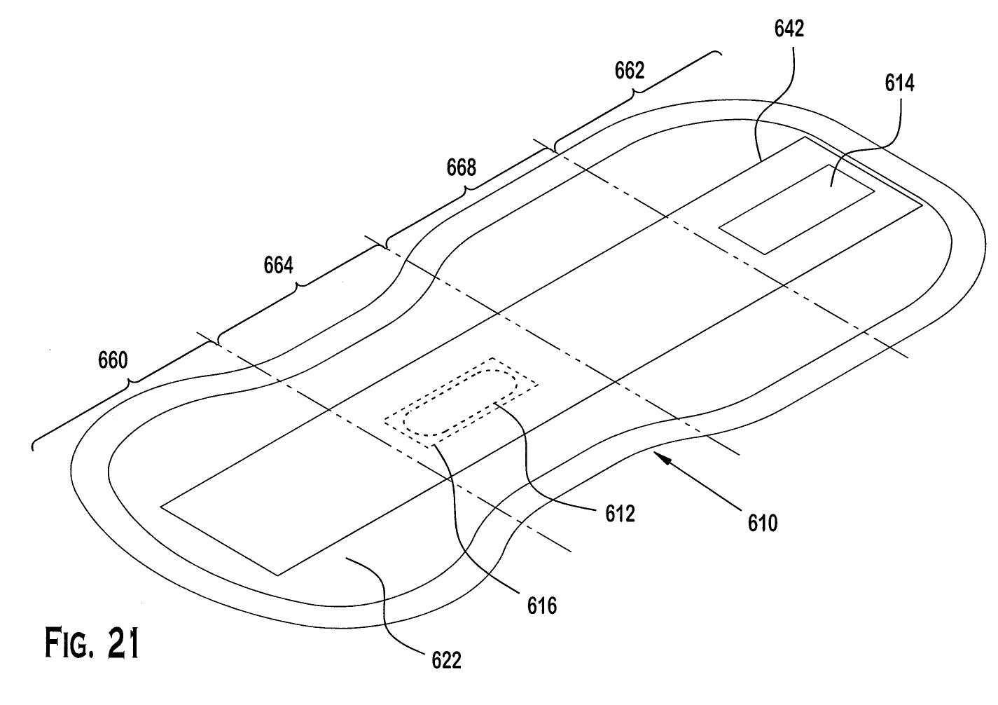

As further shown with reference to FIGS. 21 and 22, the first barrier layer 614 is attached to an outer surface of the release paper 642. Attachment may be made by way of a bead of glue 656, as shown for example in FIG. 22. However, it should be understood that the first barrier layer 614 may be attached to the release paper 642 by other means, such as by an adhesive covering the entire surface of the first barrier layer 614 facing the release paper 642, by use of a pressure sensitive adhesive, or any other securing mechanism, depending on application and design preference. Thereafter, the first barrier layer 614 and second barrier layer 616 may be sealed together, as described above.

With reference to FIGS. 17-20, operation of the opening system according to the sixth embodiment of the present invention will be described in more detail. As best shown in FIG. 20, the absorbent article 610 includes four sections, a first end section 660, a second end section 662, a third middle section 664 and a fourth middle section 668. FIGS. 17 and 22 show the absorbent article 610 in a completely folded position. In this position, the first barrier layer 614 (which is attached to the outer release paper 642) is sealed to the second barrier layer 616 thereby encapsulating the additive 612.

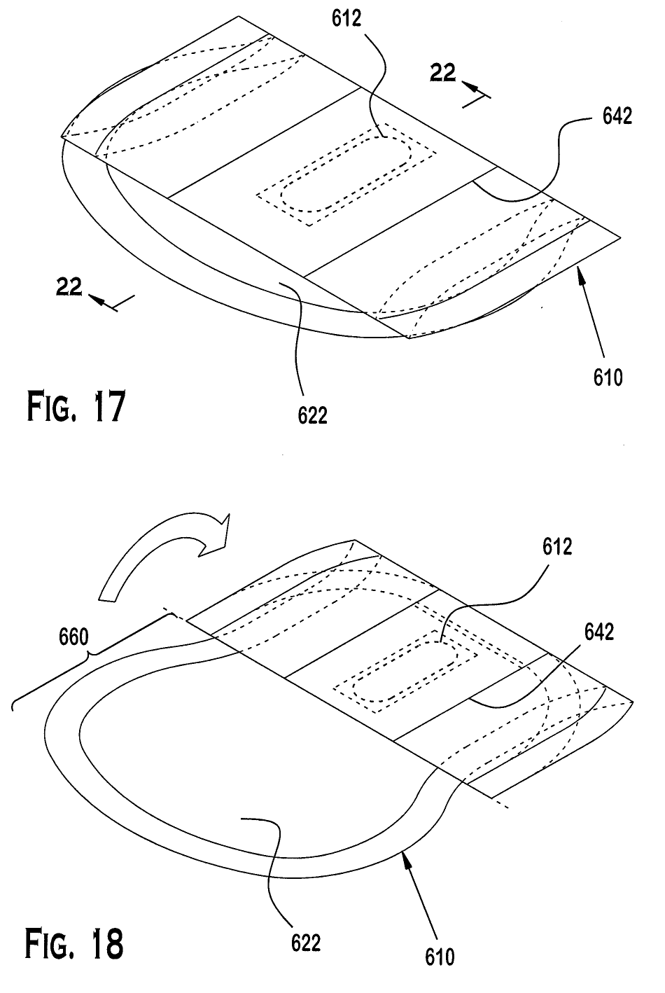

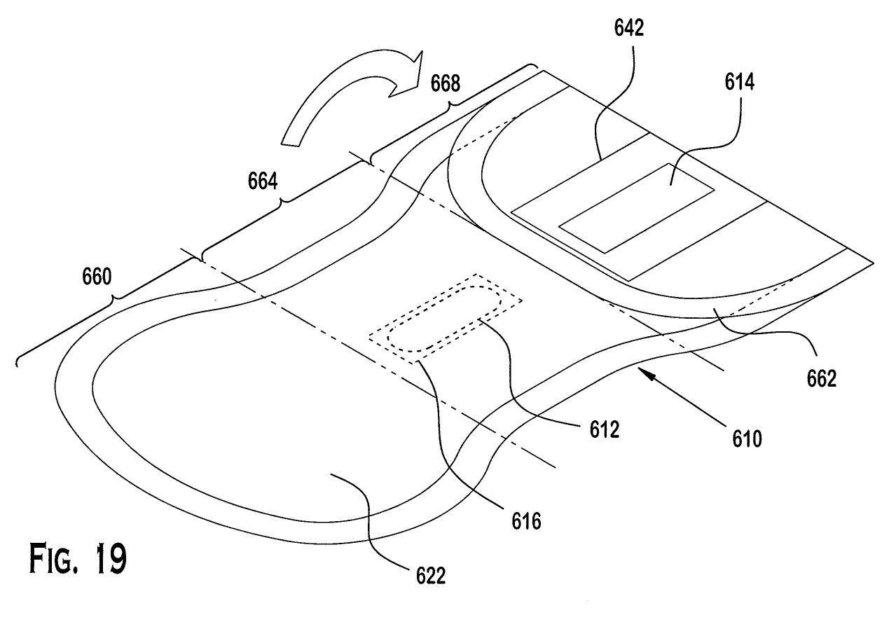

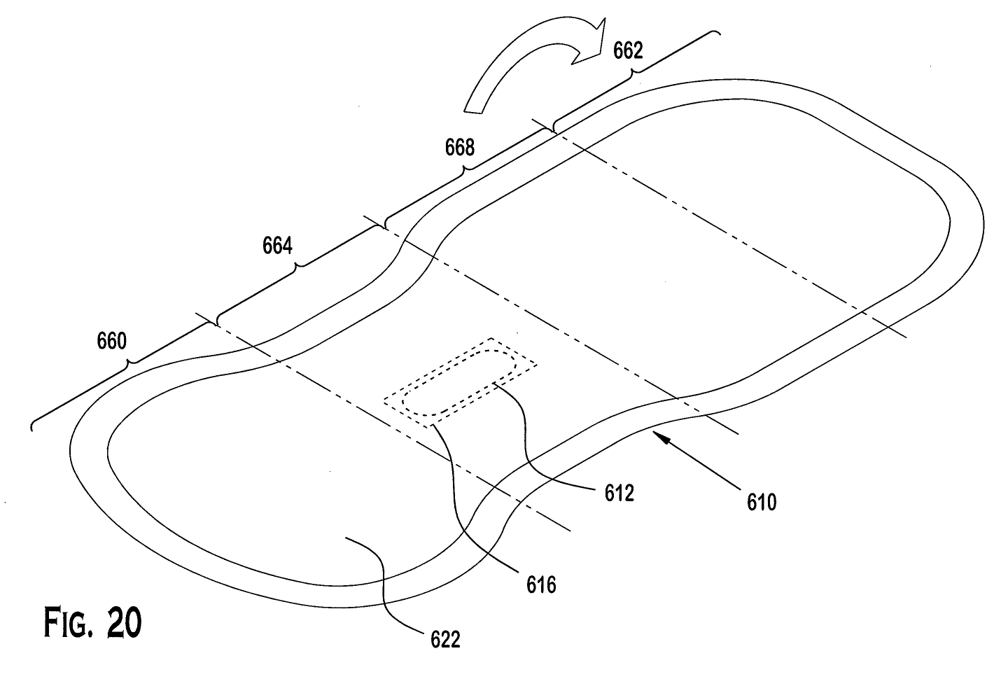

With reference to FIG. 18, the absorbent article 610 is unfolded in the direction of the respective arrow thereby revealing a first end section 660. With reference to FIG. 19, the absorbent article 610 continues to be unfolded so that the third middle section 664 is exposed. At this point, the first barrier layer 614 becomes removed automatically from the topsheet 622 of the absorbent article 610, thereby exposing the additive 612. With reference to FIG. 20, the absorbent article 610 continues to be unfolded until the entire top sheet 622 is exposed. The release paper 642 may then be removed for use.

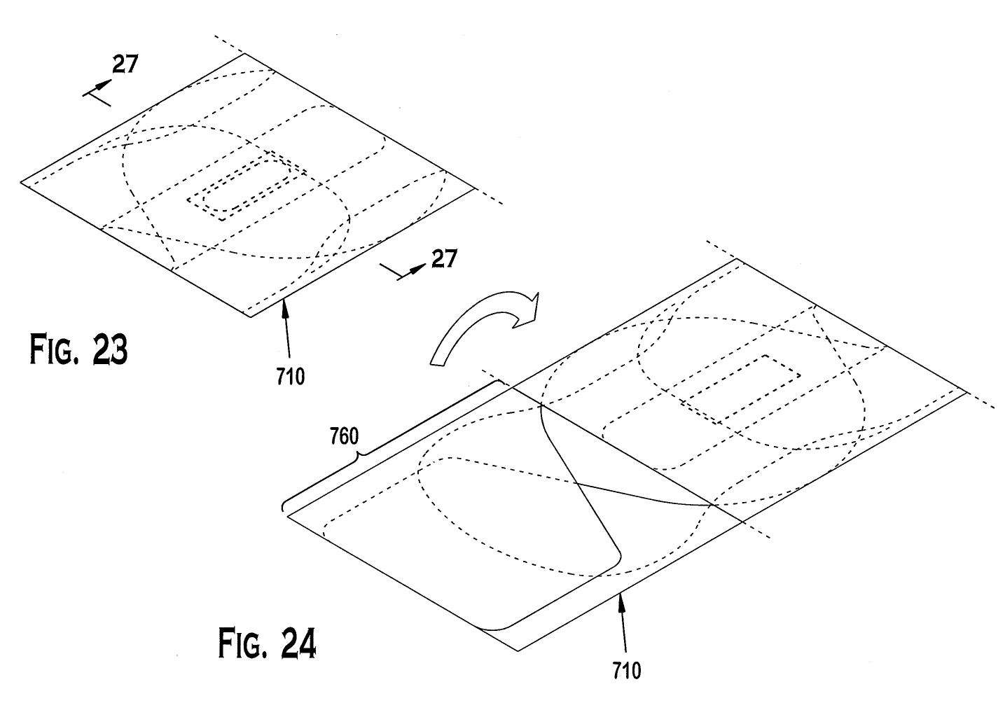

With reference to FIGS. 23-27, an absorbent article 710 according to a seventh embodiment of the present invention will be described in more detail. With reference to FIG. 27, the absorbent article 710 includes an additive 712 that is positioned between a first barrier layer 714 and a second barrier layer 716. The additive 712 corresponds to the same type of additive described above with respect to additive 112 of the first embodiment.

Like the first embodiment, the absorbent article 710 may be made from various layers of material. For example, the absorbent article 710 shown therein includes a back sheet 720 and a top sheet 722 which sandwich an absorbent core 724 therebetween. Numerous other layers may also be included, such as a glue layer 730, an additional absorbent core 732, and a transfer layer 734. However, it should be understood that the specific layers used may vary, depending on application and design preference. For example, additional layers not shown the drawings may be used, while certain layers shown in the drawings and described in the specification may be eliminated.

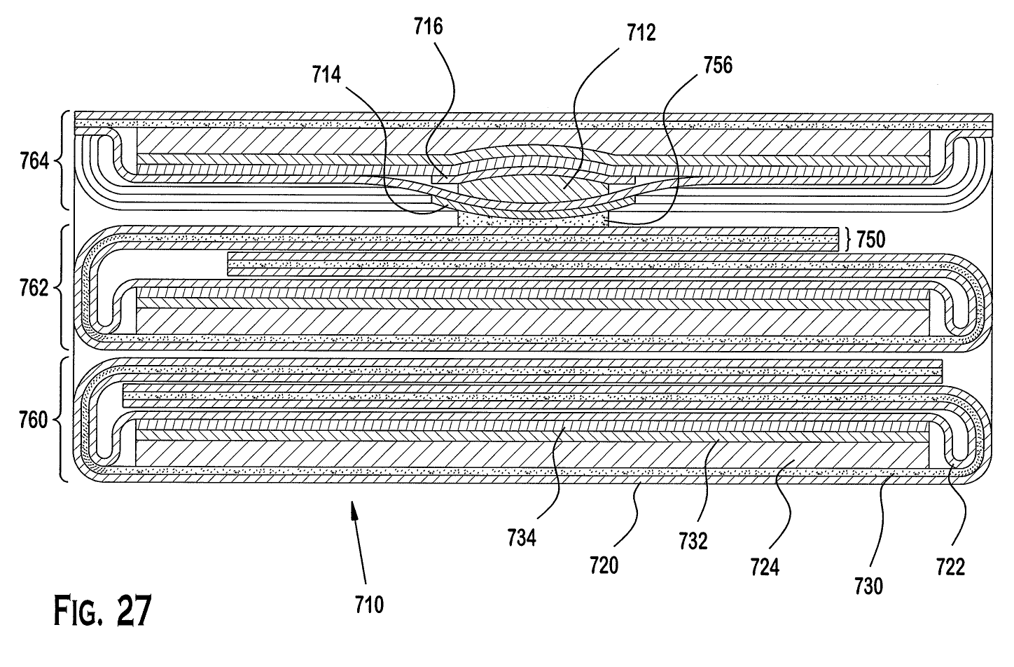

With reference to FIGS. 26 and 27, the absorbent article 710 may include side waist panels 750. Preferably, the side waist panels 750 allow the absorbent article 710 to be attached around the waist of the user, as in a diaper or adult incontinence article by an adhesive strip, or other securing mechanisms as is known in the art. In addition, a waist belt, or the like, may also be provided, depending on application and design preference.

According to the absorbent article 710 of the seventh embodiment, the first barrier layer 714 is attached to an outer portion of a side waist panel 750, as shown in FIG. 26. Attachment may be made by way of a bead of glue 756, as shown for example in FIG. 27. However, it should be understood that the first barrier layer 714 may be attached to the side waist panel 750 by other means, such as by an adhesive covering the entire surface of the first barrier layer 714 facing the side waist panel 750, by use of a pressure sensitive adhesive, ultrasonic bonding, or by any other securing mechanism, depending on application and design preference. Thereafter, the first barrier layer 714 and second barrier layer 716 may be sealed together, as described above.

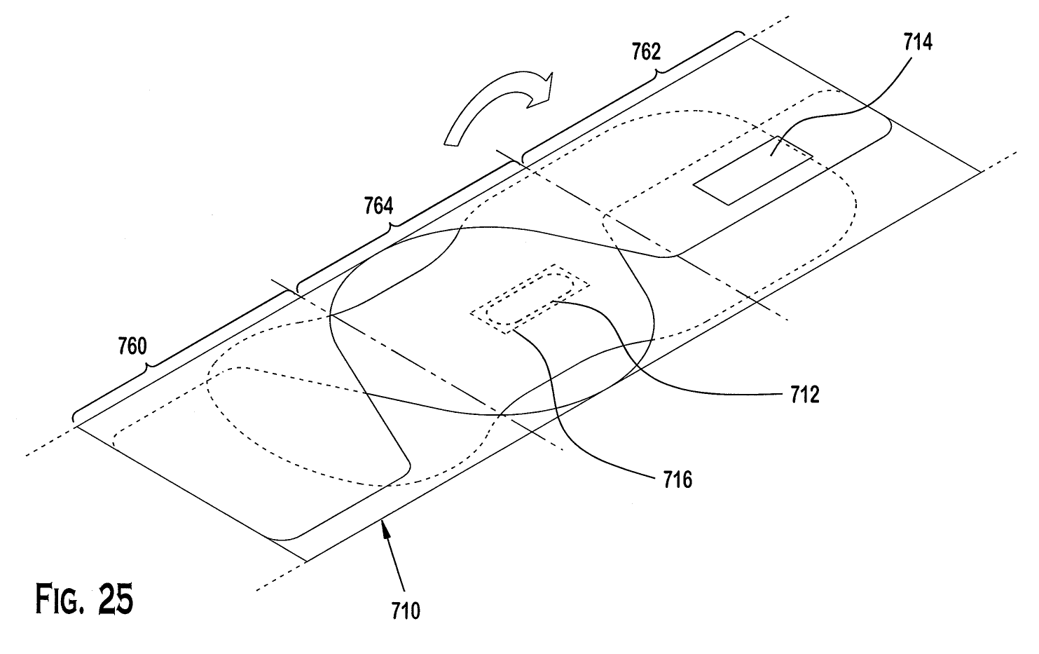

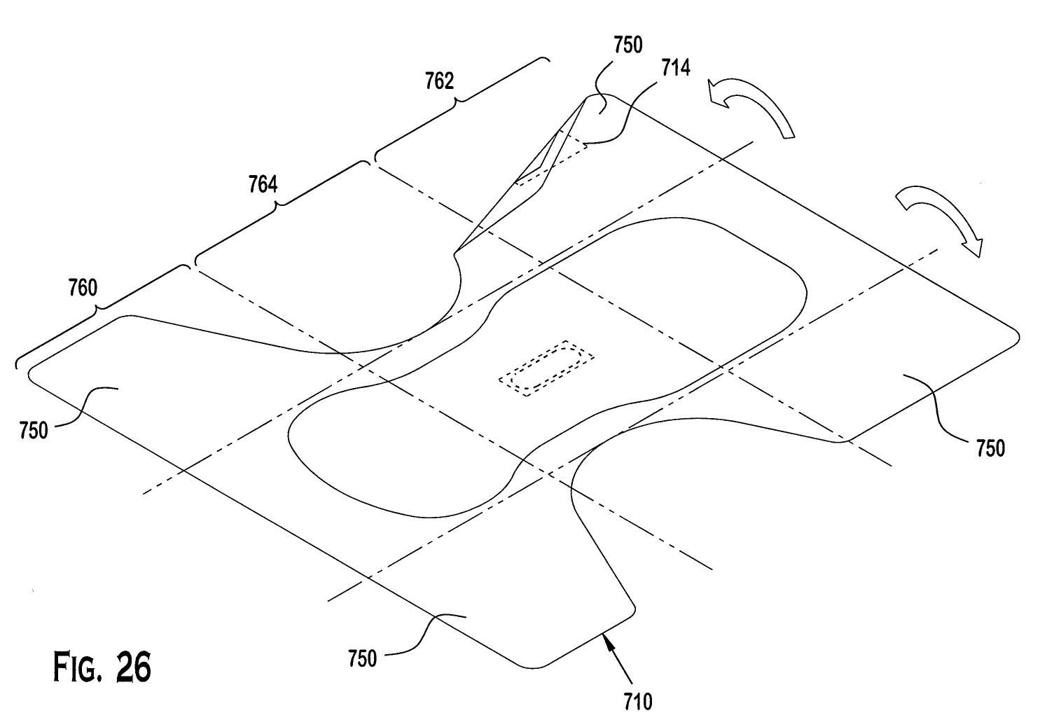

With reference to FIGS. 23-26, operation of the opening system according to the seventh embodiment of the present invention will be described in more detail. As shown in FIG. 27, the side waist panels 750 are folded inwardly, and then the absorbent article 710 is folded into three sections, a first end section 760 a second end section 762 and a middle section 764. FIG. 23 shows the absorbent article 710 is a completely wrapped position. The absorbent article 710 is unfolded in the direction of the respective arrow so that the first section 760 becomes exposed, as shown in FIG. 24. Upon opening of the second end section 762 in the direction of the respective arrow, the first barrier layer 714 becomes released from the second barrier layer 716, thereby exposing the additive 712 to moisture and the like, as shown in FIG. 25. Finally, the side waist panels 750 may be opened as shown for example in FIG. 26, and the article 710 secured around the waist of the user.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

1. An absorbent article, comprising:
a topsheet;
a backsheet;
an absorbent core disposed between the topsheet and backsheet; and
a first barrier layer and a second barrier for encapsulating an additive therebetween, wherein the second barrier layer is disposed between the topsheet and the absorbent core and the first barrier layer is disposed between the topsheet and a release paper, the first barrier layer being attached to the release paper so that the first barrier layer is removed upon removal of the release paper.

2. The absorbent article of claim 1, further including a set of wings, the release paper being attached to the set of wings.

3. The absorbent article of claim 1, wherein the first barrier layer is attached to the release paper by an adhesive or mechanical bonding.

4. The absorbent article of claim 2, further including an outer packaging, wherein the release paper is attached to an end portion of the outer packaging.

5. The absorbent article of claim 1, further including an outer packaging, wherein the release paper is attached to an end portion of the outer packaging.

6. The absorbent article of claim 1, wherein the release paper is secured to a garment facing side of the back sheet.

7. The absorbent article of claim 1, wherein the additive improves skin, health or odor of a user.

8. An absorbent article, comprising:
a topsheet;
a backsheet;
an absorbent core disposed between the topsheet and backsheet; and
a first barrier layer and a second barrier for encapsulating an additive therebetween, wherein the second barrier layer is disposed between the topsheet and the absorbent core and the first barrier layer is disposed between the topsheet and an outer packaging, the first barrier layer being attached to the outer packaging so that the first barrier layer is released from the topsheet of the article upon unfolding of the article.

9. The absorbent article of claim 8, wherein the first barrier layer is attached to a side edge of the outer packaging.

10. The absorbent article of claim 9, wherein the first barrier layer is attached to the outer packaging by an adhesive or mechanical bonding.

11. The absorbent article of claim 8, further including a set of wings, the wings being directly adhered to the outer packaging.

12. The absorbent article of claim 8, wherein the additive improves skin, health or odor of a user.

13. An absorbent article, comprising:
a topsheet;
a backsheet;
an absorbent core disposed between the topsheet and backsheet;
means for securing the article around a waist of the user; and
a first barrier layer and a second barrier for encapsulating an additive therebetween, wherein the second barrier layer is disposed between the topsheet and the absorbent core and the first barrier layer is disposed between a topsheet and the securing means, the first barrier layer being attached to the securing means so that the first barrier layer is released from the topsheet of the article upon unfolding of the article.

14. The absorbent article of claim 13, wherein the securing means include two side waist panels.

15. The absorbent article of claim 13, wherein the securing means is a waist belt.

16. The absorbent article of claim 15, wherein the first barrier layer is attached to the securing means by an adhesive or mechanical bonding.

17. The absorbent article of claim 13, wherein the first barrier layer is attached to a garment facing side of the securing means.

18. The absorbent article of claim 13, wherein the additive improves skin, health or odor of a user.
